(19) United States
(12) Patent Application Publication
(10) Pub. No.: US 2024/0029888 A1
(43) Pub. Date: Jan. 25, 2024

(54) GENERATING AND TRAVERSING DATA STRUCTURES FOR AUTOMATED CLASSIFICATION

(71) Applicant: EMED LABS, LLC, Miami, FL (US)

(72) Inventors: Nicholas Atkinson Kramer, Wilton Manors, FL (US); Michael W. Ferro, Palm Beach, FL (US); Colman Thomas Bryant, Fort Lauderdale, FL (US); John Ray Permenter, Miami, FL (US)

(21) Appl. No.: 18/357,097

(22) Filed: Jul. 21, 2023

Related U.S. Application Data
(60) Provisional application No. 63/378,931, filed on Oct. 10, 2022.
(60) Provisional application No. 63/378,916, filed on Oct. 10, 2022.
(60) Provisional application No. 63/373,643, filed on Aug. 26, 2022.
(60) Provisional application No. 63/369,287, filed on Jul. 25, 2022.
(60) Provisional application No. 63/369,041, filed on Jul. 21, 2022.

(51) Int. Cl. G16H 50/20; G06F 16/901; G16H 10/60; G16H 40/67

(52) U.S. Cl. CPC G16H 50/20; G06F 16/9024; G16H 10/60; G16H 40/67; G16H 50/30

(57) ABSTRACT

Systems, methods, and devices associated with collecting and processing user information and data to generate a data structure suitable for use by an artificial intelligence algorithm for automated classification, such as automated diagnosis and intervention of the user's condition. A custom treatment plan can be generated that is tailored to the user based on information known about the user.

### CROSS-REFERENCE TO RELATED APPLICATIONS

Any and all applications for which a foreign or domestic priority claim is identified in the Application Data Sheet as filed with the present application are hereby incorporated by reference under 37 CFR 1.57.

This application claims the benefit of U.S. Provisional Patent Application No. 63/369,041, entitled “SYSTEMS METHODS AND DEVICES FOR RISK PROFILE-DRIVEN HOLISTIC AI INTERVENTION,” filed Jul. 21, 2022, the contents of which are incorporated by reference herein in their entirety.

This application also claims the benefit of U.S. Provisional Patent Application No. 63/369,287, entitled “AI-DRIVEN DIFFERENTIAL DIAGNOSIS TOOL FOR HOLISTIC MEDICINE,” filed Jul. 25, 2022, the contents of which are incorporated by reference herein in their entirety.

This application also claims the benefit of U.S. Provisional Patent Application No. 63/373,643, entitled “SYSTEMS METHODS AND DEVICES FOR AI-ENABLED PERSONAL HEALTH,” filed Aug. 26, 2022, the contents of which are incorporated by reference herein in their entirety.

This application also claims the benefit of U.S. Provisional Patent Application No. 63/378,916, entitled “SYSTEMS METHODS AND DEVICES FOR IDENTIFICATION AND CLASSIFICATION OF COUGH OR BREATHING CHARACTERISTICS,” filed Oct. 10, 2022, the contents of which are incorporated by reference herein in their entirety.

This application also claims the benefit of U.S. Provisional Patent Application No. 63/378,931, entitled “SYSTEMS, METHODS, AND DEVICES FOR DIAGNOSTIC ORAL SCREENING,” filed Oct. 10, 2022, the contents of which are incorporated by reference herein in their entirety.

### TECHNICAL FIELD

The embodiments of the disclosure generally relate to collecting and processing user information and data to generate a data structure suitable for use by an artificial intelligence algorithm for automated classification, such as automated diagnosis and intervention of the user's condition. More specifically, the present application is directed to artificial intelligence and machine learning approaches to automatically evaluate, diagnose, and/or treat one or more user symptoms or ailments.

### BACKGROUND

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare diagnosis can solve or alleviate some problems associated with in-person diagnosis. For example, health insurance may not be required, travel to a testing site is avoided, and diagnosis can be completed at a user's convenience. However, remote or at-home diagnosis generally still depends on availability of a health provider. Accordingly, there exists a need for automated diagnosis and intervention of a user's condition to allow users to immediately obtain a diagnosis based on symptoms that they are experiencing.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

There are many undiagnosed ailments that harm quality of life, but not severely or acutely enough to by diagnosed and treated by a doctor, or for patients to even seek treatment. These ailments are often the result of many different subsystems of the human body each failing slightly rather than a single big ailment. Examples include fatigue, depression, anxiety, mental cloudiness, weakness, anhedonia, joint pains, etc.

For instance, a user may have a cough but may not want to see a medical professional, or the user may want to retrieve a diagnosis based on the cough without leaving the comfort of their own home. Or a user may have a sore throat, symptoms of COVID-19 and/or any other mouth or throat related issue or symptoms. However, the user may not want to leave their own home because the user does not feel well, or the user may not want to pay a significant amount of money to see a medical professional without an initial diagnosis.

To address such issues, a system can use a holistic approach to treating symptoms in a continual and ongoing manner via artificial intelligence. The intervention may be coupled with proctors or other external medical practitioners and may include interventions based on patient response, preference and/or risk tolerance. A system with integrated artificial intelligence and machine learning for health information collection may also be able to automatically perform tasks, such as patient intake, allowing medical professionals to spend their time performing other tasks. The system can also automatically and dynamically generate a diagnosis and a treatment plan based on the automated patient intake. The system can reduce the time required for a medical professional to diagnose a patient and develop a treatment plan further reducing the time the medical professional spends with each patient.

In specific cases, this system can also be configured to retrieve audio, image and/or movement data from a user device to automatically diagnose a user with an illness based on a cough or cough characteristics detected in the audio, image and/or movement data. The system can compare the cough or cough characteristic to cough or cough characteristics of known illnesses. Based on the diagnosis, the system can automatically provide treatment and/or management recommendations to the user. Additionally, the system can use the user device to provide treatment to the user via audio and/or vibration of the user device. The system can also be configured to capture one or more images of a mouth of the user via a camera of a user device. The system can automatically analyze the one or more images to determine an illness or medical issue of the user.

Thus, the system can use the determination of an illness or medical issue, demographic information and user health information to automatically diagnose the user with an illness or medical issue without the user leaving the user's home or seeing a medical professional in person.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 illustrates a system diagram of a telehealth proctoring platform that can be used implement an automated diagnosis system, in accordance with embodiments disclosed herein.

FIG. 2 shows an example data structure that can be generated for a user, in accordance with embodiments disclosed herein.

FIG. 3 shows an example process for determining a diagnosis and intervention, in accordance with some embodiments disclosed herein.

FIG. 4 presents a block diagram illustrating an embodiment of a computer hardware system configured to run software for implementing one or more embodiments of the systems and methods disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As used herein, the term “symptom” that may refer to any physical or mental feature which is regarded as indicating a condition of disease, particularly a feature that is apparent to the patient. The term may be used interchangeably herein with the terms “indication”, “indicator”, “sign”, “mark”, or “feature”.

As used herein, the term “condition” may refer to a medical condition, or a health impairment which results from birth, injury or disease, including mental disorder. The term may be used interchangeably herein with the terms “cause” (e.g., the cause of the experienced symptoms), “illness”, “ailment”, “disease”, “disability”, and “disorder”.

As used herein, the term “intervention” may refer to an action, solution, or approach taken to improve a situation, especially a medical condition or disorder. Examples may include a drug/medication, a diet or exercise regimen. The term may be used interchangeably herein with the terms “treatment”, “treatment plan”.

As used herein, the terms “automated diagnosis system” or “system” may refer to a set of interacting or interrelated elements (e.g., components, modules, hardware, software) that act according to a set of rules (e.g., frameworks, protocols, workflows) to form a unified whole for at least: capturing, processing, and evaluating user-associated information/data to determine symptoms; diagnosing causes of the symptoms; and determining interventions/treatments to address the causes. In some embodiments, the system may be implemented using a telehealth platform.

As used herein, the terms “digital diagnostic tool”, “differential diagnosis tool”, or “diagnostic tool” may refer to a tool that can be used by a user (e.g., on their user device) to perform some of the tasks associated with an automated diagnosis system. For example, a digital diagnostic tool may be embodied in a software application installed on the user device or a web-based application accessible via a browser on the user device. In some embodiments, a user may be able to use a digital diagnostic tool to diagnose their condition or to transmit the diagnosis to a telehealth platform.

It should be noted that the system described herein may use artificial intelligence (AI) algorithms and/or machine learning (ML) techniques to perform any tasks described herein for which AI/ML may be suitable. However, for the purpose of reducing redundancy, the tasks described herein are frequently described as being performed by the system without explicit reference to AI/ML. It should be understood that, even without explicit reference, the system may be leveraging AI/ML to provide various services to the user. For example, AI/ML may be used to process audio and/or visual data provided by the user (e.g., audio recordings of coughs or images of oral ailments); compare that user-provided data to normal baseline data (e.g., with a trained machine learning model) to determine abnormalities, symptoms, and conditions; and use that user-provided data and/or information about symptoms experienced by the user to determine likely ailments/conditions and their associated treatments. AI/ML may be used to automatically and dynamically create a list of treatments that can address or treat the ailment or symptoms experienced by a user; create a suggested set of treatments (e.g., by narrowing down the list of treatments); and order/rank treatments, such as to minimize side effects and/or additional harm. AI/ML may be used to factor clinical statistics/metrics (e.g., NNT/NNH) into treatment decisions that can be provided to clinicians.

In some embodiments, a system utilizes a multi-symptom approach to diagnose and treat a user. The system can use holistic treatment to treat one or more symptoms. In some embodiments, the system can automatically and dynamically update the holistic treatment. In some embodiments, the holistic treatment can be continual and ongoing.

In some embodiments, the user can provide information to the system about one or more conditions or ailments that the user is experiencing (and/or the associated symptoms experienced by the user). The one or more ailments can be mental ailments and/or physiological ailments. In some embodiments, the user can provide the one or more ailments (and/or the associated symptoms) via a user interface. In some embodiments, the system can retrieve the one or more ailments (and/or the associated symptoms) via an application programming interface (API). In some embodiments, the system may also receive data from fitness trackers or health monitors used by the user. In some embodiments, the system can retrieve one or more demographic inputs, environmental inputs, lifestyle inputs, and so forth. In some embodiments, these inputs can include age, race, geographical location, comorbidities, lifestyle attributes, nutrition, exercise, sleep patterns, house humidity, and so forth. These inputs can be provided directly by the user, or determined from other information made available to the system.

In some embodiments, the system can track the one or more ailments over a period of time. In some embodiments, the period of time can be predetermined (e.g., one week, one month, one year, etc.). In some embodiments, the system can track the one or more ailments with reference to the AI and/or a doctor's treatment plan. In some embodiments, the system can track the symptoms, results, and outcomes experienced by a user during and after following the AI and/or a doctor's treatment plan. In some embodiments, the system can track a location of the ailment, an intensity level, effects on energy, effects on mood, medications, supplements, and/or any other information associated with the ailment and treatment of the ailment. In some embodiments, the system can periodically prompt the user to input information. The system can prompt the user to input information based on an ailment type and/or the holistic treatment. In some embodiments, the system can determine a minimum set of information based on the ailment type and/or the holistic treatment.

In some embodiments, a frequency of the prompt can be based on the minimum set of information. The frequency can be based on an expected time for the holistic treatment to take effect and/or a volatility of the ailment. For instance, the frequency can be a period of time within the range and order of seconds, minutes, hours, days, months, years, and so forth. The period of time can be shorter for holistic treatments that can take effect in less time and/or for ailments that have a greater volatility, such as sleep quality. The period of time can be longer for holistic treatments that can take more time to take effect and/or for ailments that have a lesser volatility. For example, the user may be experiencing anhedonia while starting an anti-depressant medication that can take a month to be effective or show results, and the system can prompt the user to input information every other week. In some embodiments, the information can include a survey, a freeform description of experiences, a rating of ailments, a rating of ailment intensity, a rating of ailment frequency, a rating of treatment efficacy, and/or any other information associated with the ailment or holistic treatment.

In some embodiments, the system can request risk information from the user. The risk information can include how much the ailment bothers or affects the user, the user's risk tolerance associated with improvement of the ailment, and/or a maximum period of time the user can wait for treatment or resolution of the ailment. In some embodiments, the user's risk information may be evaluated in any determinations made about treatment/intervention. For example, the user's risk information may be used to order/rank a list of treatments, select a treatment, generate a custom treatment plan, and so forth.

In some embodiments, the system can automatically and dynamically create a list of treatments that can address or treat the ailment or symptoms experienced by a user. In some cases, the list of treatments can include approved treatments based on common medical practices. In some embodiments, the list of treatments can be ranked based on the user's risk information, and/or the information based on an ailment type, and/or the holistic treatment.

In some embodiments, the system can automatically and dynamically create a suggested set of treatments that can address or treat the ailment or symptoms experienced by a user. The suggested set of treatments can be based on the list of treatments for one or more ailments. In some embodiments, the suggested set of treatments can be based on treatments suggested by one or more medical practitioners. In some embodiments, the suggested set of treatments can include a monitoring time for each ailment or symptom. The monitoring time can be a predetermined time that the system will monitor symptoms for each ailment or treatment in the set of treatments. In some embodiments, the system can include contingency treatments for one or more of treatments in the set of treatments. The contingency treatments can include one or more treatments for the user if the ailment or symptoms do not improve with the one or more treatments of in the set of treatments. In some embodiments, the system automatically and dynamically generate and update the set of treatments and/or the contingency treatments. In some embodiments, the system can use treatments suggested by the one or more medical practitioners to update the set of treatments and/or the contingency treatments.

In some embodiments, the system may use clinical statistics, such as “Number Needed to Harm” (NNH) and “Number Needed to Treat” (NNT) from medical literature, to make decisions. These clinical statistics are often used to inform intervention decisions made by doctors. NNH is a measure of harm or adverse effects and NNT is a measure of how many patients need to be treated in order for one to benefit. Together, these statistics help physicians decide on courses of treatment. In general, lower NNT and higher NNH treatments may be preferred over those with higher NNT and lower NNH. Certain interventions (e.g., increasing exercise, increasing hydration) may have a very low NNH and a reasonably high NNT, and so these interventions are nearly “free” advice (e.g., there is a low risk of harm if one tries it). Other interventions (e.g., “take 1-theanine with your coffee to reduce anxiety”) may have high NNH and medium-to-low NNT. Despite the benefits of using the NNT/NNH ratio, many doctor decisions are frequently still made subjectively or based on anecdotes (e.g., recommending a course of treatment because it worked for the majority of their previous patients). The system disclosed herein may make it easier for clinicians to base their treatment decisions based on the NNT/NNH metrics.

In some embodiments, the system can use NNT and/or NNH to rank treatments. In some embodiments, the system can rank the treatments by weighing a likelihood of efficacy to the risk information. The system can weigh the likelihood of efficacy against the maximum period of time the user can wait for treatment or resolution of the ailment and symptom severity. In some embodiments, the system can weight the NNT and/or the NNH against the user's risk tolerance. For example, some medical literature suggests that Ashwagandha can treat anxiety; if the user has a low risk tolerance and a long maximum period of time the user can wait, Ashwagandha can be high on the list of treatments. However, if the user needs symptoms addressed as soon as possible with no regard for side effects (and the user has a high risk tolerance and a short maximum period of time that the user can wait), then Ashwagandha can be low on the list of treatments and a benzodiazepine can be high on the list of treatments.

In some embodiments, the system can determine the suggested set of treatments based on an optimal order or rank. The optimal order can start with one or more treatments that can address or treat most of or all of the user's ailments or symptoms while minimizing side effects and/or additional harm. In some embodiments, the system can use input from the medical practitioner to automatically and dynamically generate and/or update the optimal order. In some embodiments, the system can determine an underlying cause of multiple symptoms to treat the underlying cause (e.g., instead of providing a palliative remedy for the symptoms). The system may be able to rank treatment of the underlying cause as an improvement over treatment of the symptoms.

In some embodiments, the system may be able to track symptoms of a user over time. In some embodiments, the system may update a set of treatments or their ordering (e.g., to recommend a different treatment) as symptoms evolve or the overall context changes. As a specific example, consider a user experiencing fatigue, mental fog, and anhedonia. A first treatment in the optimal order can include instructing the user to hydrate and exercise. The system can track the user's symptoms throughout and after the first treatment. If the user indicates an improvement in fatigue but no improvement in mental fog and anhedonia, then the system may automatically update the optimal order and suggest a second treatment. In this example, the second treatment can be sleep tracking. The system can continue to suggest treatments and track the user's symptoms.

In some embodiments, if over the several treatments, the user still has symptoms, the system may automatically recognize that the user may have a disorder. The system can automatically recommend the user to complete a diagnostic test. In some embodiments, if the user tests positive, the system can automatically and dynamically generate a second suggested set of treatments. In some embodiments, the second suggested set of treatments can include a test-to-treat model.

In some embodiments, the system can suggest multiple treatments at a same time. The multiple treatments can be based on the NNT and the NNH to determine applicability to the ailment and risk. In some embodiments, the system can determine a rate of progress. The rate of progress can be based on an expected response time and/or the monitoring of symptoms. In some embodiments, the expected response time can be based on clinical data. In some embodiments, the suggested multiple treatments can be based on previous successful and failed treatments from other users.

In some embodiments, the system may also receive other inputs from the doctor and/or patient (e.g., severity of symptoms, demographic information, co-morbidities, acceptable time until symptom resolution, and/or risk tolerance profiles) and then generate a differential diagnosis. This provides an added benefit in that the diagnosis is not based on statistics alone and is more tailored to the individual patient. In some embodiments, the system may receive information from the doctor and/or patient such as symptoms, age, race, geographical location, co-morbidities, lifestyle attributes (e.g., family structure, hours worked per week, type of work and hobbies, etc.), nutrition (e.g., particular diets, food allergies, number of calories eaten per day, etc.), exercise routine, sleep patterns, environmental factors (e.g., location and age of home, materials to which patient is commonly exposed, job/work-related physical or mental stresses, etc.), response rate to previous treatments, number of interventions the patient is willing to adhere to before seeing results, the severity of symptoms, and the risk tolerance of the user for various side effects, etc. In some embodiments, the system may also use these inputs for determining treatment or to generate a custom treatment plan that minimizes harm and gives probabilistic outcome tracking along the way (e.g., “there is a 5% chance that an intervention plan helps within the next week, and a 98% chance that symptoms will be addressed and resolved within 6 months—let's find the least invasive approach.”).

In some embodiments, the system may perform a meta-analysis across medical literature, clinical trial data, and data from private companies (e.g., eMed). The system may assess symptoms, underlying causes, NNT/NNH, demographic information, etc. for each intervention supported by the system (which can be dynamically expanded). In other words, the system may determine the efficacies of a particular intervention for treating associated conditions/ailments, the symptoms associated with those treatable conditions, and so forth. In some embodiments, the system may store this data and these relationships in a database, which can be used in diagnosing a user and suggesting an intervention.

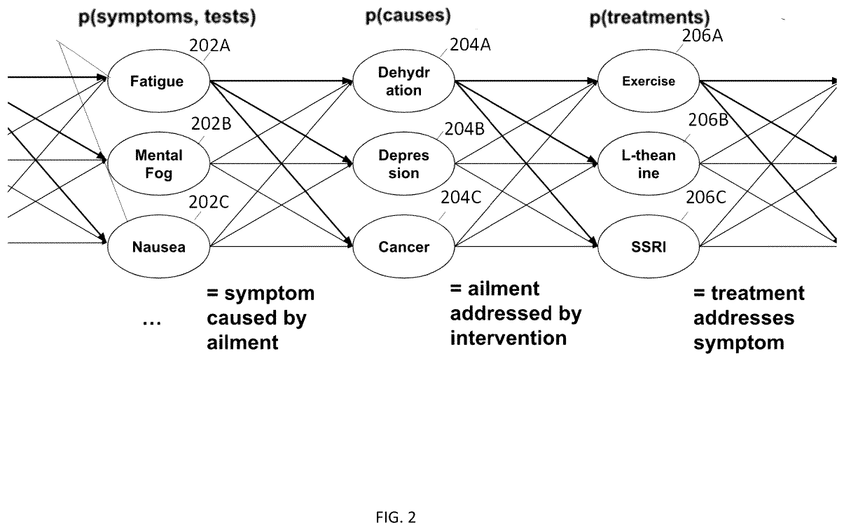

To summarize, in some embodiments, the system may be able to receive various user-associated data and information. This can include descriptions of symptoms; audio/visual data captured by the patient of their symptoms; doctor-provided inputs and patient-provided inputs (e.g., demographic information, co-morbidities, acceptable time until symptom resolution, and/or risk tolerance profiles). The system may be able to determine a probability outcome space for what underlying causes are likely to cause the patient's symptoms and provide a ranked list of intervention options to address the underlying causes based on each intervention's likelihood of efficacy. (An example data structure and approach for making these determinations is discussed in connection with FIG. 2.) In some embodiments, the system may also be able to use the user-associated data and information to generate a differential diagnosis and/or intervention that is more tailored to the individual patient and not solely based in statistics alone. The system may also be able to factor in or codify NNT and NNH metrics and rank them against the acceptability of likely outcomes (e.g., a cost function).

As a specific example that demonstrates many of these concepts together, consider a user experiencing depression and fatigue during the summer. The user may also be experiencing cystic acne. In this example, the user may have a low risk tolerance and may be willing to wait a long time for depression, fatigue, and cystic acne to be treated. The system can determine a cause of the cystic acne has a 3 percent chance to be age related hormone changes, 5 percent chance to be hygiene, 22 percent to be acute allergies, 67 percent to be mold exposure related to endocrine changes, and 2 percent unknown. The treatment for the user's symptoms can include antibacterial soap, topical spironolactone, and antifungal medications. Antibacterial soap can have a low risk profile and a low chance of effectiveness. The topical spironolactone can have a medium risk profile and a high chance of effectiveness. The antifungal medications can have a low risk profile and a medium chance of effective ness. Based on a determined chance of each cause, the low risk tolerance and the long maximum period of time the user can wait, the system may recommend the antibacterial soap and the antifungal medications, with 1-week check-ins over a period of 3 months. After 1 month, the user may see mild improvement. The system can incorporate the improvement into updating the suggested set of treatments and can recommend stopping the antibacterial soap. After 2 months, the user may see no improvement. The system can incorporate the no improvement into updating the suggested set of treatments. The system can incorporate data about the antibacterial soap and the antifungal medications, and determine the soap was not effective and the antifungal medications should have shown more results. The system can update the suggested set of treatments, and suggest the user use topical spironolactone with 2-week check-ins. After 6 weeks, the user may see significant improvements with the cystic acne and may have depression symptoms. The system may suggest the user continue to use the topical spironolactone and the system can create a new set of treatments for the depression symptoms. The user may have a different risk tolerance and/or a different maximum period of time the user can wait. The system can suggest the user go to a separate mental wellbeing department for the treatment of the depression symptoms.

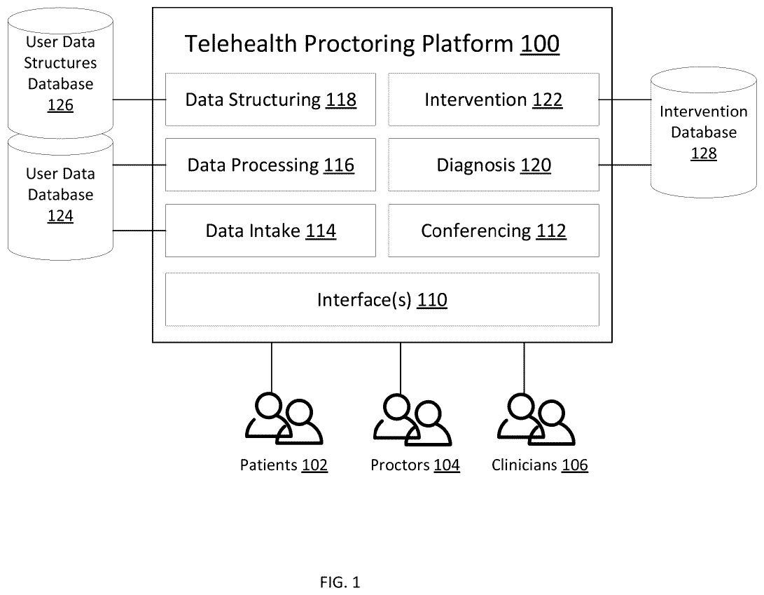

Turning now to the figures, FIG. 1 illustrates a system diagram of a telehealth proctoring platform that can be used implement an automated diagnosis system and provide automated diagnosis and intervention for its users (e.g., patients of telehealth services), in accordance with embodiments disclosed herein. More specifically, it illustrates a system diagram of a telehealth proctoring platform 100.

It should be noted that functionality of the telehealth proctoring platform 100 may be described and/or shown as components, modules, and systems. These illustrations and descriptions of the various components of the telehealth proctoring platform 100 are provided for the purpose of facilitating ease of understanding. In practice, the functionality of the telehealth proctoring platform 100 does not need to be siloed or delineated in the same manner. For instance, one or more of the components may be optional, used together, or combined. Furthermore, one or more of the components may be separately located (e.g., with a third-party or at an endpoint) or their corresponding functionality may be performed at various locations. For example, the interfaces 110 may include user interfaces displayed to a patient through a website or web-based application (with the user interface data provided by a server on the backend), or alternatively, the user interfaces could be displayed by an application running on the patient's user device (and that application may or may not be part of the telehealth proctoring platform 100).

It also should be noted that the term “users” may sometimes refer to the patients 102 (who are proactively using the telehealth proctoring platform 100 for telehealth services), especially in the context of seeking a diagnosis or intervention for their condition. However, the term “users” may also refer to any of the patients 102, proctors 104, and/or clinicians 106 (who could all be considered users in the literal sense since they all interact with the telehealth proctoring platform 100). The term “supervising users” may refer to the proctors 104 and/or clinicians 106, who may be able to supervise patients 102 as they self-administer diagnostic tests and medication, inquire about ailments/conditions that they may be experiencing, perform tasks in accordance with a medical treatment plan, and so forth. In some embodiments, the term “supervising users” may also refer to an AI chatbot or system that interacts with the patients 102 in a humanlike manner despite not actually being human.

In some embodiments, the patients 102, proctors 104, and/or clinicians 106 may be able to interact with the telehealth proctoring platform 100 through one or more interfaces 110 associated with the telehealth proctoring platform 100. These interfaces 110 may include various user interfaces and/or application programming interfaces (APIs) depending on the implementation of the telehealth proctoring platform 100. For example, in some embodiments, one or more of the patients 102, proctors 104, and/or clinicians 106 may access the telehealth proctoring platform 100 via their user device (not shown) by accessing an application installed on their user device or a web-based application, which will provide various user interfaces that can be interact with. In some embodiments, one or more of the patients 102, proctors 104, and clinicians 106 may access an application installed on their user device or a web-based application, and that application may communicate with the telehealth proctoring platform 100 via an API. In some embodiments, the interfaces 110 may be embodied in a digital diagnostic tool (e.g., a frontend of an automated diagnosis system), which can be installed or accessed on a user device in order to receive a diagnosis based on symptoms and/or user-provided data.

Generally, the patients 102 may include any person that has availed themselves of telehealth services via the telehealth proctoring platform 100. The patients 102 may include any person looking to receive a diagnosis based on their symptoms and/or user-provided data. As a general matter, the telehealth proctoring platform 100 can be used to provide virtual proctoring and supervision for self-administration of medical procedures, medications, and diagnostic tests; for check-ins scheduled as part of a medical treatment plan; and for any questions or assistance associated with a generated diagnosis or intervention. The patients 102 may include any persons with known ailments or proclivities towards developing certain medical conditions. The telehealth proctoring platform 100 can be used to allow telemedicine practitioners (e.g., proctors 104 and/or clinicians 106) specifically possessing experience and training on those ailments and conditions to be assigned to monitor the person and immediately intervene when the person needs assistance.

The patients 102 may utilize various user devices to connect to the telehealth proctoring platform 100 and attend virtual proctoring sessions or avail themselves of telehealth services. Non-limiting examples of the user devices may include a mobile or other handheld device (e.g., cell phone, smartphone, tablet, laptop, etc.). In some embodiments, the patients 102 may be able to interact with the telehealth proctoring platform 100 through one or more interfaces 110 associated with the telehealth proctoring platform 100. These interfaces 110 may include various user interfaces and/or application programming interfaces (APIs) depending on the implementation of the telehealth proctoring platform 100. For example, in some embodiments, the patients 102 may have direct access to the telehealth proctoring platform 100 on their user devices (e.g., via an installed application, web-based application, website, etc.), which can display user interfaces that the patients 102 can interact with.

In some embodiments, the patients 102 may have user tracking devices that continuously monitor and collect user status data (e.g., any kind of data associated with the user, such as GPS location data). In some cases, a patient's user device may also be a user tracking device (e.g., a smartphone may track the GPS location of the patient). In some cases, the user tracking devices may be able to directly send the data to the telehealth proctoring platform 100. In some cases, the user tracking devices may be able to indirectly send the data to the telehealth proctoring platform 100, such as by providing the data to a user device for transmission to the telehealth proctoring platform 100. These user tracking devices may include health tracking devices, such as health sensors and/or health monitors, that continuously monitor and collect the patient's real-time health data (e.g., health information, bioindicators, physiological data, vitals, etc.). Some non-limiting examples of health sensors and/or health monitors may include remote patient monitoring (RPM) devices, point-of-care sensing devices, and wearable technology such as smart watches and health/fitness trackers. For instance, wearable technology is often used to monitor a user's health. Wearables can be used to collect data on a user's health including: heart rate, calories burned, steps walked, blood pressure, release of certain biochemicals, time spent exercising, seizures, physical strain, body composition, and water levels. Additionally, wearable technology may be used to monitor glucose, alcohol, lactate, blood oxygen, breath monitoring, heartbeat, heart rate and its variability, electromyography (EMG), electrocardiogram (ECG) and electroencephalogram (EEG), body temperature, pressure (e.g. in shoes), sweat rate or sweat loss, and levels of uric acid and ions—e.g. for preventing fatigue or injuries or for optimizing training patterns. Additionally, wearable technology may be used to measure mood, stress, and health; measure blood alcohol content; measure athletic performance; monitoring how sick the user is; detect early signs of infection; perform long-term monitoring of patients with heart and circulatory problems by recording an electrocardiogram; perform days-long continuous imaging of diverse organs via a wearable bioadhesive stretchable high-resolution ultrasound imaging patch (e.g. a wearable continuous heart ultrasound imager); perform sleep tracking, monitor cortisol levels for measuring stress; and measure relaxation or alertness, e.g. to adjust their modulation or to measure efficacy of modulation techniques.

The proctors 104 may include medical professionals (e.g., physician, nurse, nutritionist, health coach, and/or the like) that can monitor, supervise, and provide instructions or real-time guidance to the patients 102 (e.g., in a virtual proctoring session facilitated by the telehealth proctoring platform 100). The proctors 104 may be able to perform various roles for many different situations and contexts. For example, a proctor 104 may be able to supervise a patient 102 performing a medical diagnostic test to verify adherence to proper test procedure. The proctor 104 may be able to ensure test result authenticity (e.g. that the test results have not been swapped or tampered with) or even provide suggestions or interpretations of the diagnostic test results. More specifically, a proctor 104 could virtually meet with a patient 102 to go over instructions for a lateral flow test to detect COVID-19 and then assist the patient 102 with interpreting the results of the lateral flow test. In another similar example, the proctor 104 may be able to supervise a patient 102 while they self-administer a medication (e.g., inject themselves with a drug) to provide instructions and guidance on how to administer the medication correctly (e.g., the exact location the drug should be injected, the correct dosage, and so forth).

In some embodiments, a proctor 104 may be able to virtually meet with a patient 102 to go over instructions for a medical treatment plan, to monitor the patient's progress during the medical treatment plan, to confirm the patient is adhering to the plan, and to review any symptoms/results experienced by the patient during the medical treatment plan. In some embodiments, the proctors 104 may be trained or experienced in handling varying ailments or medical conditions, and a patient 102 having a known ailment or medical condition can be assigned one or more proctors 104 that specifically possess experience and training for it. Those assigned proctors 104 may be able to monitor that patient 102 to detect symptoms of, or the worsening of, the ailment/condition. In some cases, those proctors 104 may be able to monitor the patient 102 through virtual proctoring sessions (e.g., live video streams, telehealth conferences, etc.).

The proctors 104 may utilize various user devices to connect to the telehealth proctoring platform 100 and perform telemedicine functions. In some embodiments, the proctors 104 may be able to interact with the telehealth proctoring platform 100 through one or more interfaces 110 associated with the telehealth proctoring platform 100. These interfaces 110 may include various user interfaces and/or application programming interfaces (APIs) depending on the implementation of the telehealth proctoring platform 100. For example, in some embodiments, the proctors 104 may have direct access to the telehealth proctoring platform 100 via their user device (e.g., via an installed application, web-based application, website, etc.), which can display user interfaces that the proctors 104 can interact with to perform telemedicine functions (e.g., attend a virtual proctoring session).

The clinicians 106 may refer to any doctor that has contact with, and direct responsibility for, a patient and is capable of approving or modifying a patient's medical treatment plan. There may be some overlap between the roles/functions of the proctors 104 and the clinicians 106 (e.g., both may be considered telemedicine practitioners and engage in telehealth conferencing with patients). In some cases, there may not be much distinction between the two or the distinction may be difficult to discern. However, in some cases, the clinicians 106 may be able to make additional decisions that many of the proctors 104 would not be able to make, such as modifications to a patient's medical treatment plan or prescribed medication.

The clinicians 106 may utilize various user devices to connect to the telehealth proctoring platform 100 and perform telemedicine functions. In some embodiments, the clinicians 106 may be able to interact with the telehealth proctoring platform 100 through one or more interfaces 110 associated with the telehealth proctoring platform 100. These interfaces 110 may include various user interfaces and/or application programming interfaces (APIs) depending on the implementation of the telehealth proctoring platform 100. For example, in some embodiments, the clinicians 106 may have direct access to the telehealth proctoring platform 100 via the user devices (e.g., via an installed application, web-based application, website, etc.), which can display user interfaces that the clinicians 106 can interact with to perform telemedicine functions (e.g., attend a virtual proctoring session, review a patient's health records, make modifications to a prescribed medication or medical treatment plan, etc.).

In some embodiments, the telehealth proctoring platform 100 can include a conferencing system or module 112. In some embodiments, the conferencing module 112 can be configured to connect a patient 102 and a proctor 104 in a telehealth or virtual proctoring session. In some embodiments, the conferencing module 112 can be configured to connect the patient 102 and the proctor 104 via a video conferencing session, such as via live video (e.g., over the Internet of cellular communication network). In some embodiments, the conferencing module 112 can be configured to facilitate video calls, audio calls and/or telemedicine calls. In some embodiments, the patient 102 may access the conferencing module 112 via their user device, and the proctor 104 may access the conferencing module 112 via their respective user device (e.g., a proctor device).

In some embodiments, the conferencing module 122 may be configured to establish a live, virtual proctoring session between a patient and a proctor. For example, it may enable a patient 102 to provide to a proctor 104 a live video feed of the patient 102 (e.g., for providing updates on their symptoms). In some cases, a patient may be assigned to a specific proctor or a group of proctors in advance (e.g., to a particular medical professional or group of medical professionals). In some cases, the patient may be assigned to one of the proctors 104 based on availability (e.g., who is available when the patient initiates a proctoring session), and/or based on personal considerations (e.g., the patient's sex, gender, age, co-morbidities, dietary preferences, and so forth).

Virtual proctoring sessions may be scheduled (e.g., at regular intervals as part of a medical treatment plan) or provided on-demand (e.g., to confer with a patient needing immediate assistance with an ailment/condition, etc). In some cases, a patient may be the initiator of scheduled or on-demand virtual proctoring sessions. In other cases, the telehealth proctoring platform 100 itself or a proctor 104 may be the initiator of scheduled or on-demand virtual proctoring sessions. For example, a schedule of regular and periodic check-ins for a medical treatment plan can be established for the patient. As another example, a proctor 104 evaluating the patient's health information may catch troubling indications (e.g., high blood pressure) associated with the patient's ailment/condition and initiate a virtual proctoring session to bring it to the patient's attention.

In some embodiments, the telehealth proctoring platform 100 can include a data intake system or module 114. The data intake module 114 may be configured to collect various kinds of information or data associated with a user (e.g., patient 102). In some cases, a user may provide the information or data directly (e.g., by filing out a form, sending files, etc.), whereas in other cases, the information or data may be collected without direct user involvement (e.g., from various devices, from a proctor 104 or clinician 106, and so forth). In some embodiments, collected user information or data for a user may be stored in a user data database 124 or processed (e.g., by the data processing module 116) before being stored in the user data database 124.

In some embodiments, the data intake module 114 may be configured to collect user symptom information. For example, the user can provide information to the system about one or more symptoms associated with a condition/ailment that the user is experiencing, such as via a user interface.

In some embodiments, the data intake module 114 may be configured to collect user risk information. For example, the system can request risk information from the user (e.g., via a user interface). The risk information can include how much the symptoms/ailment bothers or affects the user, the user's risk tolerance associated with improvement of the ailment, and/or a maximum period of time the user can wait for treatment or resolution of the ailment.

In some embodiments, the data intake module 114 may be configured to obtain user multimedia data captured by a user using one or more devices and/or sensors. For example, a user can capture one or more images, videos, and/or audio recordings with their user device. As a more specific example, the images and/or videos can include a user's face, eyes, mouth, and/or any other portion of the user's body. The audio recording can include a user's cough, a user's breathing, a user speaking, a user's heartbeat, and/or any other noise produced by the user. In some embodiments, the nature of any requested user multimedia data may be based on the user symptom information; the user's symptoms may be used to select based on relevancy the user multimedia data, the devices and/or sensors, and any instructions provided to the user. For example, if the user reported symptoms with their skin, then images or video (and not audio) can be captured of the user's skin.

In some embodiments, the data intake module 114 may be configured to retrieve user health statistics data from one or more devices or sensors. For example, the user may have health sensors and/or health monitors that continuously collect the user's health statistics to send to the telehealth proctoring platform 100. Accordingly, the data intake module 114 may retrieve bioindicators, physiological data, vitals, or other quantitative data associated with the user from any health sensors or monitors, such as a thermometer, a fitness watch, a smartwatch, a heart rate monitor, blood pressure cuffs, and so forth. In some embodiments, the system can automatically determine the devices or sensors (and also the user health statistics) relevant to the user's symptoms based on the user-provided user symptom information and/or user multimedia data (e.g., the one or more images, videos, and/or audio recordings).

In some embodiments, the data intake module 114 may be configured to obtain additional user health information considered relevant, which can include user demographic information, user lifestyle information, user environmental information, and so forth. For instance, this group of information may include the user's age, race, geographical location, comorbidities, lifestyle attributes (e.g., family structure, hours worked per week, type of work and hobbies, job/work-related physical or mental stresses, etc.), nutrition (e.g., particular diets, food allergies, number of calories eaten per day, etc.), exercise, sleep patterns, environmental factors (e.g., location and age of home, house humidity, materials to which patient is commonly exposed, etc.), responses to previous treatments, and so forth. This information can be provided directly by the user or determined from other information made available to the system.

In some embodiments, the telehealth proctoring platform 100 can include a data processing system or module 116. The data processing module 116 may be configured to take the various kinds of information or data collected for a user and process it into a more-useable format. In some embodiments, this processing may be tailored to the user's symptoms or the requirements of the algorithms for diagnosis/intervention. For example, in some embodiments, the system may receive an audio recording of the user and the data processing module 116 may resample the audio recording, apply filters to the audio recording, convert the contents of the audio recording to text, and so forth, to place the data into a format more suitable for evaluation by an AI algorithm to determine a diagnosis or intervention.

In some embodiments, the telehealth proctoring platform 100 can include a data structuring system or module 118. The data structuring module 118 may be configured to take the various kinds of information or data associated with a user (e.g., patient 102), such as the information/data in the user data database 124, in order to generate a data structure for the user that can aid in automatically generating diagnoses and interventions. More information is provided about these data structures in connection to FIG. 2. In some embodiments, the data structure for a user may be saved in a user data structures database 126.

In some embodiments, the telehealth proctoring platform 100 can include a diagnosis system or module 120. The diagnosis system or module 120 may be configured to take the various kinds of information or data associated with a user (e.g., the information/data stored in user data database 124) and/or the data structure for the user (e.g., stored in the user data structures database 126) and generate a diagnosis for the cause of the symptoms the user is experiencing. For instance, the diagnosis module 120 may traverse the data structure for the user (as described in connection with FIG. 2) to determine a likely condition associated with the symptoms the user is experiencing. In some embodiments, the diagnosis module 120 may also determine one or more interventions/treatments for addressing the diagnosed condition, such as by traversing the data structure for the user. In some embodiments, the diagnosis module 120 may also rank or order interventions, provide suggested interventions to a user, and/or generate a custom intervention plan or treatment plan for the user based on the selected intervention.

In some embodiments, the telehealth proctoring platform 100 can include an intervention system or module 122. In some embodiments, the intervention module 122 may be integrated together with the diagnosis module 120. In some embodiments, the intervention module 122 may perform functions such as determining one or more interventions/treatments for addressing the diagnosed condition of a user, ranking or ordering interventions, providing suggested interventions to a user, generating a custom intervention plan or treatment plan for the user based on the selected intervention, and so forth. However, in some embodiments, the intervention module 122 may be primarily concerned with the execution and implementation of an intervention based on a user's health information and condition.

In some embodiments, the intervention module 122 can be configured to automatically implement various interventions based on a patient's health information and condition. In some embodiments, the intervention module 122 can be configured to present to a telemedicine practitioner a report that indicates possible ailments/conditions that a patient may have and the input data and analysis that was used in making that determination. In some embodiments, the intervention module 122 can be configured to present to a telemedicine practitioner (e.g., via a graphical user interface) a patient's health information and/or different options for implementing various interventions.

In some embodiments, the telehealth proctoring platform 100 can include an intervention database 128. In some embodiments, the intervention database 128 may specify the various interventions supported by the system (which can be dynamically expanded). The intervention database 128 may contain data associated with each intervention, such as the conditions/ailments treatable by the intervention, efficacies of the intervention for treating each associated condition/ailment, the symptoms associated with each of those treatable conditions, and so forth. In some embodiments, this data can be used by the data structuring module 118 to generate or update the data structure associated with a user. In some embodiments, this data can be used by the diagnosis module 120 and/or to the intervention module 122 to diagnose a user with a condition and suggest one or more interventions.

FIG. 2 is a recurrent tripartite connected directed acyclic graph (“DAG”) illustrating an example data structure that can be generated for a user for use with embodiments of the differential diagnosis tool disclosed herein.

The DAG may be interpreted as a graph with nodes (e.g., 202A-C, 204A-C, 206A-C), with edges (e.g., lines or arrows) that connect various pairs of nodes in the graph. As shown in the figure, the left side of the graph has nodes that represent every possible symptom or indication (e.g., 202A-C), the middle of the graph has nodes that represent every possible underlying cause or condition (e.g., 204A-C), and the right of the graph has nodes that represent every possible intervention or treatment (e.g., 206A-C). In other words, this can be thought of as three different categories or layers of nodes—a first layer of nodes representing different symptoms or indications, a second layer of nodes representing different causes or conditions, and a third layer of nodes representing different interventions or treatments.

In some embodiments, edges may connect each of the nodes of the first layer to each of the nodes of the second layer. In some embodiments, edges may connect each of the nodes of the second layer to each of the nodes of the third layer. For instance, leaving each symptom node (e.g., 202A, 202B, 202C) and entering each underlying cause node (e.g., 204A, 204B, 204C) is an edge (e.g., a line or arrow) representing the probability the symptom (corresponding to the symptom node) is indicative of that cause (corresponding to the cause node). Leaving each underlying cause node (e.g., 204A, 204B, 204C) and entering each treatment node (e.g., 206A, 206B, 206C) is an edge representing the probability that the proposed treatment (corresponding to the treatment node) addresses the underlying cause (corresponding to the cause node).

In some embodiments, there may be edges leaving the nodes of the third layer (e.g., the treatment nodes). In some embodiments, these edges may conceptually loop back to the nodes of the first layer (e.g., the symptom nodes). For instance, the edges leaving each treatment node (e.g., 206A, 206B, 206C) may be interpreted as entering each symptom node (e.g., 202A, 202B, 202C), with each edge representing the likelihood that the treatment (corresponding to the treatment node) addresses the symptom (corresponding to the symptom node) and/or how effectively the treatment addresses the symptom.

In some embodiments, each node is “stateful” and contains information such as the presence of a symptom as a percentage, a currently recommended treatment, an amount of time passed since beginning the recommended treatment, and the like. This allows traversal through the graph as though it were dynamic rather than static or fixed. Traversing the graph may be visualized as a vertical line looping through the three node categories while repeatedly simulating a treatment plan. In some embodiments, a graph may be generated for a user or patient and updated based on symptoms experienced by the user and/or various other information provided by the user. The graph may be traversed in order to determine a likely cause (e.g., corresponding to a particular cause node) for the symptoms experienced by the user, to determine at least one treatment (e.g., corresponding to at least one treatment node) for the likely cause, and so forth.

In some embodiments, the system may complete a random sample consensus (“RANSAC”) type approach, where hundreds of thousands of random simulations are performed with arbitrary or heuristically guided decisions at each stage. During each of these simulations, three events may occur. First, the simulated patient may accumulate either relief of symptoms or accumulation of negative effects. Second, medical tests may be performed to change the probabilities between the first and second layer of nodes (e.g., to rule out or decrease the probability associated with one or more underlying causes). Third, treatments may be introduced and/or modified and/or stopped.

In some embodiments, the system may select at least one treatment/intervention based on a cost function. In some embodiments, the system may also generate a custom treatment plan based on a cost function. For example, when the RANSAC algorithm reaches diminishing returns on the cost function (e.g., the system has maximized probability of success and minimized probability of harm according to the patient's risk tolerance profile stops improving by much), the system may select the top one or more treatment plans with accompanying graphs of the risks/improvements/milestones along the way.

In some embodiments, selected treatment plans may be provided to a doctor to discuss with the patient. Alternatively or additionally, the treatment plans may be directly presented to the patient (e.g., through a telehealth platform, such as the one offered by eMed) if the interventions have sufficiently low risk (e.g., as may be determined by setting a predetermined NNH threshold, symptom intensity threshold, severity threshold, etc.).

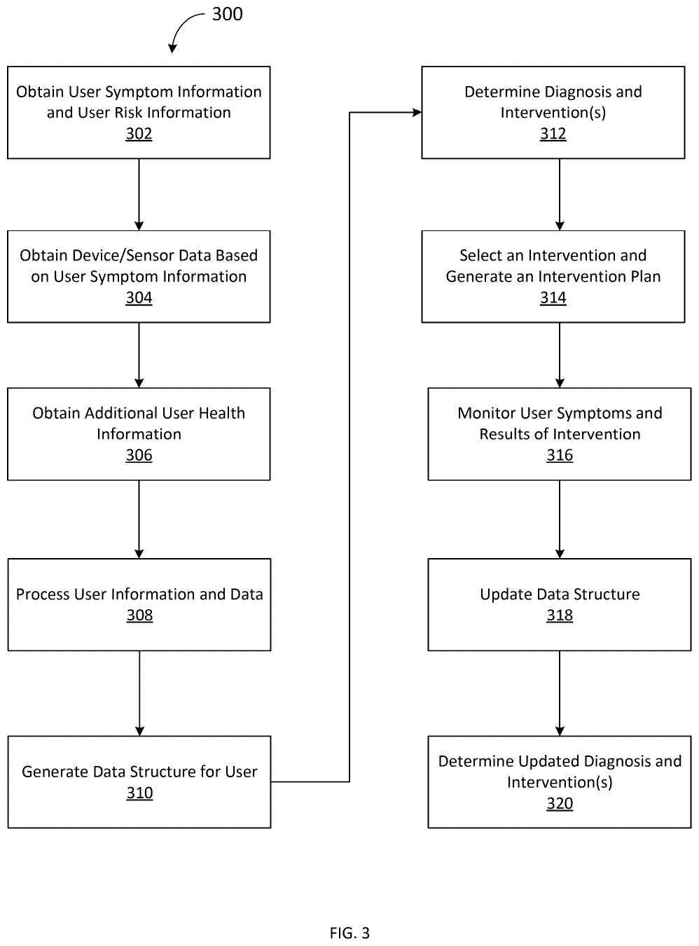

FIG. 3 shows an example process 300 for determining a diagnosis and intervention, in accordance with some embodiments disclosed herein. Some of the blocks may be optional or performed in a different order than what is depicted.

At block 302, the system may obtain user symptom information and user risk information from a user. For example, the user can provide information to the system about one or more symptoms associated with a condition/ailment that the user is experiencing, The user risk information may be directed to how much the symptoms/ailment bothers or affects the user, the user's risk tolerance associated with improvement of the ailment, and/or a maximum period of time the user can wait for treatment or resolution of the ailment.

At block 304, the system may obtain data from devices and/or sensors based on the user symptom information. For instance, the system may obtain user multimedia data (e.g., one or more images, videos, and/or audio recordings) captured by a user using one or more devices and/or sensors. The system may also obtain user health statistics data from one or more devices or sensors, such as bioindicators, physiological data, vitals, or other quantitative data. The nature of the user multimedia data and the user health statistics data collected may depend on the user's symptoms, such that only relevant data is collected.

At block 306, the system may obtain any additional user health information considered relevant, which can include user demographic information, user lifestyle information, user environmental information, and so forth. In some embodiments, this information may be used in the generation of a custom treatment plan that is tailored to the user.

At block 308, the system may process any of the information and data collected to place the data into a format more suitable and useable for evaluation by an AI algorithm to determine a diagnosis or intervention. For example, the user's heart signal or heart rate data may be processed to determine if there is an elevated heart rate, which can be used as an additional user symptom.

At block 310, the system may generate a data structure for the user based on the available information and data on the user, particularly the user's symptoms.

At block 312, the system may determine a diagnosis (e.g., a likely condition causing the user's symptoms) and one or more interventions for treating the condition. In some embodiments, the data structure may be generated at block 310 and then traversed as described in connection with FIG. 2 in order to determine a cause and one or more treatments. In some embodiments, the system may use different approaches to determine a diagnosis and one or more interventions. For example, the system may have a trained machine learning model that can determine when a body part is afflicted by a particular condition based on an image of the body part, and the model can be applied to an image of the user's body part to determine if that particular condition is present.

At block 314, a particular intervention may be selected. For example, the system may automatically determine the most optimal intervention based on various criteria, such as the information/data known about the user (e.g., the user is allergic to these medications) and a cost function (e.g., to maximize probability of success and minimize probability of harm). The user or a clinician may also have input in selecting an intervention. In some embodiments, a intervention/treatment plan may be generated for the user. This treatment plan may be tailored for the user based on various criteria, such as the selected treatment, the information/data known about the user, a cost function, and so forth. The user or a clinician may also have input in designing the intervention plan.

At block 316, as the user follows the treatment plan, the system will monitor the user's symptoms and results. For example, the user may be scheduled to check-in and provide updates on a periodic basis.

At block 318, the system may update the data structure for the user based on the user's symptoms and results with the treatment plan. For example, with the additional information, certain causes for the symptoms can be ruled out. The data structure may be updated to reflect this. In some cases, the previous determined cause can even be ruled out, which may require a new determination of an updated diagnosis and intervention at block 320.

In some embodiments, the system can use AI and ML to evaluate symptoms of a user. The system can diagnose a user with one or more injuries, diseases, infections, or any other health related illness. In some embodiments, the system can order one or more prescriptions and/or recommend one or more treatment plans.

In some embodiments, the system can be a website, web application, a mobile application, or any other software. In some embodiments, the system can include a graphical user interface (GUI) displayed on a user device. The system may retrieve, via a user input, user intake information. In some embodiments, the user intake information can include demographic information (e.g., name, age, gender, etc.), symptom information (e.g., sore throat, stuffy nose, headache, etc.) or any other common intake information.

In some embodiments, the system can receive or prompt the user to input audio information. The system can automatically and dynamically convert in real-time, or substantially real-time, the audio information to text. In some embodiments, the system can display the text to the user. In some embodiments, the system can display the text as part of a message chain displayed on the user device.

In some embodiments, the user can input one or more images, videos, and/or audio recordings into the system. The system can use computer vision (CV) and/or AI to analyze the one or more images, videos, and/or audio recordings of the user to obtain health information and generate a diagnosis. For example, the images and/or videos can include a user's face, eyes, mouth, and/or any other portion of the user's body. The audio recording can include a user's cough, a user's breathing, a user speaking, a user's heartbeat, and/or any other noise produced by the user. In some embodiments, the system can analyze the one or more images, videos, and/or audio recording to obtain the user's vitals.

In some embodiments, during collection of the one or more images, videos, and/or audio recordings, the system can automatically and dynamically instruct a user to indicate one or more areas of discomfort and/or pain. For example, the one or more areas of discomfort and/or pain can be a location of where the user's head hurts from a headache. The system can use CV to automatically identify a location of the one or areas of discomfort and/or pain. The system can automatically and dynamically incorporate the location into the health information to generate a diagnosis.

In some embodiments, the system can automatically instruct a user to perform one or more actions during collection of the one or more images, videos, and/or audio recordings. In some embodiments, the one or more actions can be based on the user intake information. In some embodiments, the one or more actions can include the user coughing, opening the user's mouth, capturing an image of an affected area of the user's body and/or any other user actions. In some embodiments, the system can instruct the user to use a tongue depressor or any other diagnostic tool during collection of the one or more images, videos, and/or audio recordings. In some embodiments, the system can request a user to input a period of time the user has experienced the symptoms for. In some embodiments, the input can be entered by the user via the user device, or the input can be an audio recording. In some embodiments, the system can automatically associate each period of time with each of the symptoms based on the input, the one or more images, videos, and/or audio recordings. In some embodiments, based on the user intake information, the system can dynamically ask a user one or more diagnostic questions. In some embodiments, follow up diagnostic questions can be based on a user response to a previous diagnostic question.

In some embodiments, the system can automatically retrieve data from one or more secondary devices or sensors. The secondary devices or sensors can include a thermometer, a fitness watch, a smartwatch, a heart rate monitor, blood pressure cuffs, and/or any other devices or sensors. The system can automatically determine the secondary devices or sensors relevant to the user's symptoms based on the user intake information and/or the one or more images, videos, and/or audio recordings.

In some embodiments, if the user's symptoms are related to the user's skin, the system can instruct the user to select a skin tone from a display of a plurality of skin tones. The selected skin tone can be the skin tone that is closest to a skin tone of the user. In some embodiments, the system can use the selected skin tone to adjust one or more camera settings of the user device. The camera settings can include exposure, white balance, ISO, shutter speed, aperture, and/or any other camera settings. In some embodiments, the system can use the selected skin tone to automatically and dynamically calibrate or update the AI and CV algorithms for analysis of one or more images of the user's skin.

In some embodiments, the system can display one or more models of a body on the GUI. The system can request the user to input on the one or more models one or more locations of the user's symptoms.

In some embodiments, the system can obtain health information from one or more diagnostic tests. The one or more diagnostic tests can include COVID-19 tests, pregnancy tests, UTI tests, STD tests, strep throat tests, flu test, and/or any other diagnostic test. In some embodiments, the system can automatically order and/or deliver the one or more diagnostic tests to the user based on the user intake information and/or the health information.

In some embodiments, the system can use AI to automatically generate a diagnosis based on the user intake information and/or the health information. In some embodiments, the diagnosis can include a summary of the intake information, the health information, and/or collection of the intake information and health information. In some embodiments, the summary can include health statistics (e.g., blood pressure, heart rate, respiration rate, body temperature, etc.). The system can automatically, based on the demographic information, determine a scale for each of the health statistics. The scale can include a range of value generally considered too low, normal, and too high. The system can automatically display the health statistics on the scale associated with each of the health statistics. In some embodiments, the diagnosis can include one or more of the one or more images, videos, and/or audio recordings. In some embodiments, the diagnosis can include the one or more models of one or more locations of the user's symptoms, and/or any other information collected by the system.

In some embodiments, based on the diagnosis, the system can automatically order and deliver one or more diagnostic tests to the user.

In some embodiments, the system can use AI and ML to automatically generate a treatment or treatment plan for the user based on the diagnosis. In some embodiments, the treatment can include a medication, and the system can automatically recommend a specific medication, a dose, and/or a medication schedule or frequency. In some embodiments, the system can automatically request contact information from the user. The contact information can include an email address, a phone number, an address, and/or any other contact information. In some embodiments, the system can automatically send the user the diagnosis, the summary, and/or the treatment or treatment plan. In some embodiments, the system can send the diagnosis, the summary, and/or the treatment or treatment plan to a medical professional for approval before the system send the diagnosis, the summary, and/or the treatment or treatment plan to the user. In some embodiments, the system can automatically send a prescription for the medication to a pharmacy for pick up by the user or delivery to the user.

### Digital Diagnostic Tools

Block 304 of FIG. 3 describes how the system may obtain user multimedia data (e.g., one or more images, videos, and/or audio recordings). In some embodiments, the system can include one or more digital diagnostics tools. In some embodiments, the one or more digital diagnostic tools can collect or retrieve data from the user regarding the user's health, such as by collecting or retrieving audio and/or image data from the user. In some embodiments, a digital diagnostic tool can use a microphone and/or a camera of a user device to collect or retrieve the audio and/or image data. In some embodiments, a digital diagnostic tool can collect or retrieve the audio and/or image data via a data collection process. In some embodiments, the digital diagnostic tool can prompt the user to perform one or more steps of the data collection process. In some embodiments, the digital diagnostic tool can prompt the user via a computer graphic display on a display of the user device. In some embodiments, the digital diagnostic tool can display one or more images and/or augmented reality graphics. In some embodiments, the digital diagnostic tool can prompt the user via audio generated by a speaker of the user device or transmitted to a secondary audio device, such as a Bluetooth speaker, headphones, and/or any other audio device.

In some embodiments, a user can provide preliminary information (e.g., user symptom information) to the system. The preliminary information can include demographic information, symptom information, and/or any other information relevant to the user or a user's health. The system can automatically determine a digital diagnostic tool that is relevant to the user based on the preliminary information. In some embodiments, the system and/or the telehealth platform may display or provide the relevant digital diagnostic tool to the user. The one or more digital diagnostic tools can relate to various symptom such as coughing, eye inflammation, burry vision, ear pain, allergies, blood sugar imbalance, headaches, anxiety, cognitive changes, and/or any other symptoms.

In some embodiments, a digital diagnostic tool can use artificial intelligence and/or machine learning to automatically and dynamically determine a possible illness of the user by comparing the audio data, the image data, and/or data retrieved from the one or more other sensors to data or data templates of the one or more known illnesses. In some embodiments, the digital diagnostic tool can use artificial intelligence and machine learning to automatically and dynamically improve a diagnosis accuracy of the digital diagnostic tool by comparing the initial diagnosis to results of one or more diagnostic tests.

### Identification and Classification of Cough or Breathing Characteristics

As a particular example, the system may be used for cough detection and analysis for the purpose of diagnosis. In some embodiments, the system can use acoustic epidemiology to determine an illness of the user. The system can analyze retrieved audio and/or image data to automatically determine qualities or characteristics of the user's cough that are associated with one or more known illnesses. In some embodiments, the qualities or characteristics of the user's cough can include a dryness or wetness of the cough, a pitch, a number of coughs, a frequency of the cough, and/or any other characteristics of a cough. In some embodiments, the one or more known illnesses can include COVID-19, influenza, a cold, bronchitis, tuberculosis, pneumonia, fluid detection, whooping cough, lung cancer, sleep apnea, chronic conditions such as asthma, smoking, or other exposure related illnesses, smoke inhalation (e.g. from a house fire or a wildfire), and/or any other illnesses with identifiable cough qualities or characteristics. In some embodiments, the system can automatically determine an illness the user has based on the cough qualities or characteristics of the user's cough.

In some embodiments, a digital diagnostic tool can be used to retrieve audio data of the user's cough. In some embodiments, the user can hold the user device with the microphone near or up to the user's mouth. In some embodiments, the user can cough into the microphone one or more times so the digital diagnostic tool can retrieve audio data of the user's cough. In some embodiments, the user can cough three times. In some embodiments, the user can cough as deeply as the user can. In some embodiments, the digital diagnostic tool can use one or more other sensors of the user device and the audio data to determine when a user coughs so the digital diagnostic tool can determine a portion of the audio data associated with the user's cough. In some embodiments, the one or more other sensors can be an acceleration sensor, a camera, or any other sensor of the user device that can detect movement. In some embodiments, the digital diagnostic test can use the one or more other sensors and the audio data to determine a cough severity. In some embodiments, the digital diagnostic tool can automatically prompt the user in real-time or substantially real time, and/or provide real-time or substantially real-time feedback if the digital diagnostic tool does not detect one or more coughing or breathing sounds and/or motion of the user or user device.

In some embodiments, the user can place the user device on the user's chest. In some embodiments, the user can lay down prior to or after placing the user device on the user's chest. In some embodiments, the user can breathe deeply, and the digital diagnostic tool can retrieve audio data of the user breathing via the microphone of the user device. In some embodiments, the digital diagnostic tool can prompt the user to move to one or more body positions (e.g., standing up, sitting, laying on stomach, etc.). In some embodiments, the digital diagnostic tool can prompt the user to move the user device to one or more locations of the user's body while the user is at each of the one or more body positions. The digital diagnostic tool can retrieve audio data at each of the one or more locations and each of the one or more body position. The digital diagnostic tool can use artificial intelligence and/or machine learning to automatically and dynamically determine an illness of the user by comparing the audio data with data or data templates of the one or more known illnesses. In some embodiments, the digital diagnostic tool can use artificial intelligence and/or machine learning to analyze the audio data an detect one or more breathing abnormalities of the user. The one or more breathing abnormalities can include wheezing, rhonchi, crackles, stridor, and/or any other breathing abnormalities.

In some embodiments, the digital diagnostic tool can play one or more sounds or tones from the speaker of the user device. The digital diagnostic tool can retrieve audio data from the microphone of the user device and compare the audio data to the one or more sounds or tones to detect changes in the one or more sounds or tones. The digital diagnostic tool can use the changes in the one or more sounds or tones to determine whether the user has fluid or other irregularities in the lungs of the user.

In some embodiments, the digital diagnostic tool can use the determined possible illness of the user, the one or more breathing abnormalities, and/or the fluid or other irregularities in the lungs of the user to determine an initial diagnosis. The initial diagnosis can indicate a likely illness of the user. The likely illness of the user can include an illness the digital diagnostic tool determines the user most likely has based on the determined possible illness of the user, the one or more breathing abnormalities, and/or the fluid or other irregularities in the lungs of the user.

In some embodiments, the diagnosis can include one or more follow up recommendations. The follow up recommendations can include one or more diagnostic tests, an appointment with a medical professional, and/or suggested care instructions. If the likely illness is an illness that requires treatment the digital diagnostic tool can automatically generate a prescription and/or a link to prescription ordering platform. In some embodiments, the digital diagnostic tool can automatically generate and transmit periodic reminders to the user. The periodic reminders can include reminders to take one or more diagnostic tests and/or reminders to take a follow-up test if the one or more diagnostic tests are positive. In this way, the digital diagnostic tool can use the one or more diagnostic tests to confirm or verify the initial diagnosis.

In some embodiments, if the digital diagnostic test cannot determine a likely illness with a confidence level above a predetermined threshold, the digital diagnostic test can provide the follow up recommendations to the user.

In some embodiments, the digital diagnostic tool can include one or more passive functions. In some embodiments, the user can grant or deny an application with the digital diagnostic tool permission to use the one or more passive functions. The one or more passive functions can include a listening function. The listening function can use the microphone of the user device to listen for and detect coughs of the user at any time. The digital diagnostic tool can track trends or changes in a user illness of the user over time. In some embodiments, the trends or changes can include cough severity, cough frequency, and/or cough productivity. The digital diagnostic tool can use the trends or changes to determine whether a treatment is working, and the user illness is treated effectively such that the user illness is clearing up. In some embodiments, if the user illness is not clearing up the digital diagnostic tool can automatically alert the user and suggest additional treatments to the user.

In some embodiments, the digital diagnostic tool can retrieve data from one or more ancillary user devices. The one or more ancillary user devices can include a smartwatch, a fitness tracker and/or any other device that can monitor biometric data of the user. In some embodiments, the one or more ancillary user devices can detect an oxygen level of the user. The digital diagnostic tool can connect to the one or more ancillary devices via a wireless connection or a wired connection. In some embodiments, the user can input data from the one or more ancillary devices into the digital diagnostic tool. In some embodiments, the digital diagnostic tool can use the data from one or more ancillary devices to track the trends or changes in the user illness.

In some embodiments, the digital diagnostic tool can automatically and dynamically recommend one or more actions to the user based on the trends or changes in the user illness. The one or more actions can include additional tests, alternative treatments, rest, scheduling an appointment with a medical professional, visiting the emergency room, and/or any other action related to treatment or management of an illness.

In some embodiments, the digital diagnostic tool can automatically transmit alerts, metrics, and/or graphics to the use based on detected illnesses of a plurality of other users. In some embodiments, the detected illnesses can include illnesses of a plurality of other users of the digital diagnostic tool, or data/metrics retrieved from public databases. In some embodiments, the graphics can include a map that displays one or more locations or areas where there are detected illnesses. In some embodiments, the digital diagnostic tool can automatically transmit to the user one or more alerts when a contagious disease is detected within a predetermined distance of a location of the user. In some embodiments, the digital diagnostic tool can transmit the one or more alerts when a predetermined threshold of a population has the contagious disease.

In some embodiments, a database of cough characteristics associated with positive or negative test results can be automatically updated over time. The database of cough characteristics can be used as the data or data templates of the one or more known illnesses as described in various embodiments herein.

In some embodiments, the digital diagnostic tool can include one or more treatments. The digital diagnostic tool can use one or more functions of the user device to provide the one or more treatments to the user. In some embodiments, the diagnostic tool can play loud audio and/or vibrate a vibration motor of the user device to alter or break up fluid, phlegm or other material in the user's lungs. In some embodiments, the user can place the user device on the user's chest. In some embodiments, to improve an effectiveness of the one or more treatments, the digital diagnostic tool can prompt the user to move to one or more body positions (e.g., standing up, sitting, laying on stomach, etc.). In some embodiments, the digital diagnostic tool can prompt the user to move the user device to one or more locations of the user's body while the user is at each of the one or more body positions.

### Visual Screening and Diagnosis

As another particular example, the system may be used for the detection and diagnosis of various conditions (e.g., oral conditions) that can be identified through visual analysis. In particular, a digital diagnostic tool may be able to capture and analyze visual data (e.g., images, video, etc.) of the user's afflicted body part or anatomical feature. In some embodiments, the digital diagnostic tool may be able to analyze visual data (e.g., images, video, etc.) captured by the user. For example, the user may be able to capture images and/or video of parts of their body, such as by using a camera on their user device. In some cases, the user may be prompted to capture images and/or video of the part of their body that is associated with the condition (e.g., the part of the body that the user suspects is abnormal).

In some embodiments, the digital diagnostic tool can determine a size of each of the one or more anatomical features. The digital diagnostic tool can retrieve camera information from the user device. The camera information can include a focal length, an aperture, an ISO, a shutter speed, a camera resolution, a frame rate, a zoom level, distance from a lens of the camera to the user and/or the anatomical feature, and/or any other camera information. The digital diagnostic tool can use the camera information and image data of the one or more images to determine the size of the one or more anatomical features.

In some embodiments, the digital diagnostic tool can analyze an appearance of a mouth and/or throat of a user for diagnosis and/or evaluation of the user. In some embodiments, the digital diagnostic tool can capture one or more images of the mouth or throat of a user and analyze image data in the one or more images to diagnose the user with an illness based on the appearance of the mouth or throat. The digital diagnostic tool can use one or more cameras of a user device to capture the one or more images.

In some embodiments, the digital diagnostic tool can capture one or more images of an outside of the mouth of the user, an inside of the mouth of the user, and/or the throat of the user. In some embodiments, the one or more images can be one or more photos. In some embodiments, the one or more images can be one or more video streams.

In some embodiments, the digital diagnostic tool can use artificial intelligence, machine learning and/or computer vision algorithms to identify one or more anatomical features or landmarks of the mouth and/or throat of the user. The one or more anatomical features or landmarks can include teeth, gums, tonsils, tongue, hard palate, soft palate, uvula, papillae and/or any other anatomical feature of the mouth.

In some embodiments, the digital diagnostic tool can determine a size of each of the one or more anatomical features. The digital diagnostic tool can retrieve camera information from the user device. The camera information can include a focal length, an aperture, an ISO, a shutter speed, a camera resolution, a frame rate, a zoom level, distance from a lens of the camera to the user and/or the anatomical feature, and/or any other camera information. The digital diagnostic tool can use the camera information and image data of the one or more images to determine the size of the one or more anatomical features. In some embodiments, the user can place a reference marker of a predetermined size in the mouth of the user, so the reference marker is in the one or more images. The digital diagnostic tool can use the predetermined size as a reference to determine the size of the one or more anatomical features. In some embodiments, the reference marker can on a tongue depressor and/or any other piece of material sized to fit in the mouth.

In some embodiments, the digital diagnostic tool can automatic ally determine a presence of one or more anatomical features. The digital diagnostic tool can analyze the image data to determine if the user has tonsils, wisdom teeth, and/or any other anatomical feature. In some embodiments, the digital diagnostic tool can analyze the image data to determine an alignment or spacing of the teeth of the user to determine whether the user requires braces or any other tooth aligners.

In some embodiments, the digital diagnostic tool can compare the size each of the one or more anatomical features to a predetermined size range. The predetermined size range can include a range of size considered to be standard or healthy. In some embodiments, the predetermined size range can be based on user information. The user information can include a size of the mouth, a height of the user, a weight of the user, a gender, an age, past medical procedures, past illnesses, one or more medications, a health history of the user, and/or any other user information that can affect or determine a standard of healthy size of the one or more anatomical features.

In some embodiments, the digital diagnostic tool can compare the size and/or a location of each of the one or more anatomical features on a first side of the mouth to a corresponding anatomical feature on a second side of the mouth. For example, the digital diagnostic tool can compare the size and/or location of tonsils, corresponding teeth on each side of the mouth, etc. In some embodiments, the digital diagnostic tool can determine a symmetry score for the one or more anatomical features and the corresponding anatomical features. In some embodiments, the symmetry score can be a ratio of a symmetry of the size and/or location of the one or more anatomical features and the corresponding anatomical features. For example, if an anatomical feature and a corresponding anatomical feature have a same size and a same location, the symmetry score can be 1:1. If the corresponding anatomical feature is missing or the digital diagnostic tool cannot identify the corresponding anatomical feature, the symmetry score can be 1:0. In some embodiments, if the symmetry score indicates asymmetry more than a predetermined threshold between the first side of the mouth and the second side of the mouth, the digital diagnostic tool can request additional information from the user. In some embodiments, the digital diagnostic tool can base the diagnosis of an illness at least on the symmetry score.

In some embodiments, the digital diagnostic tool can analyze the image data to determine colors, color patterns, textures and/or texture patterns of the one or more anatomical features. For example, the digital diagnostic tool can determine that soft tissue of the mouth (e.g. gums, tongue, roof of mouth, etc.) is a pink color, however, at least a portion of the mouth near the tonsils has one or more white spots. Specific colors, patterns and/or textures on certain portions of the mouth can indicate and/or be correlated with one or more illnesses. In some embodiments, the digital diagnostic tool can determine the colors, color patterns, textures and/or texture patterns via artificial intelligence, machine learning and/or computer vision algorithms. In some embodiments, the digital diagnostic tool can determine relative colors and/or absolute colors. In the embodiments where the digital diagnostic tool can determine absolute colors, the user can place a reference image or color grid on a tongue depressor, or any other material, in the mouth. The digital diagnostic tool can use the reference image or color grid to determine a white balance or base color adjustment for color calibrations or pixel color labeling. In some embodiments, the digital diagnostic tool can access the camera information and retrieve a determined white balance from the camera of the user device.

In some embodiments, the digital diagnostic tool can compare the one or more images and/or the image data to one or more images and/or image data of positive cases of one or more illnesses to diagnose the user with an illness. For example, illnesses such as strep throat or tonsilitis can be determined from redness, white patches and/or dark red petechiae. The digital diagnostic tool can compare the one or more images and/or the image data to the one or more images and/or the image data with redness, white patches and/or dark red petechiae.

In some embodiments, the digital diagnostic tool can assist the user with positioning of the user device and/or camera such that a quality of the one or more images is above a predetermined threshold. The digital diagnostic tool can provide visual and/or audio prompts to instruct the user where to move the user device and/or the camera relative to the mouth. The visual and/or audio prompts can include graphics, augmented reality, displayed words, audio words, and/or sounds. In some embodiments, the digital diagnostic tool can automatically determine, based on the camera information and/or image data, when the user device and/or camera is in a proper location for capturing the one or more images. In some embodiments, the digital diagnostic tool can automatically capture the one or more images when the user device and/or camera is in the proper location. In some embodiments, the digital diagnostic tool can display the one or more images to the user and question the user to confirm that one or more of the anatomical features are visible in the one or more images. In some embodiments, the user can select to retake the one or more images if one or more of the anatomical features are not visible and/or if the image quality is below the predetermined threshold.

In some embodiments, the user and/or the digital diagnostic tool can determine the image quality. The digital diagnostic tool can automatically analyze the one or more images and determine if a sharpness, a brightness, a white balance, and/or an angle or position of the camera relative to the mouth are each below a predetermined threshold. If one or more of the sharpness, the brightness, the white balance, and/or the angle or position of the camera relative to the mouth are below the predetermined threshold, the digital diagnostic tool can automatically recapture the one or more images or prompt the user to recapture the one or more images.

In some embodiments, one or more of the one or more images can be captured at an angle and/or position of the camera relative to the mouth that is different from an angle and/or position of the camera relative to the mouth when a first image was captured.

In some embodiments, the digital diagnostic tool can analyze each of the one or more images separately or the digital diagnostic tool can stitch the one or more images together to form a composite image. The digital diagnostic tool can analyze the composite image using artificial intelligence, machine learning and/or computer vision algorithms. In some embodiments, the one or more images can include one or more frames from a video captured by the camera of the user device. The digital diagnostic test can automatically analyze the video to determine one or more frames to analyze.

In some embodiments, the digital diagnostic tool can analyze image data in the one or more images to diagnose the user with COVID-19, strep throat, and/or tonsilitis. In some embodiments, the digital diagnostic tool can analyze image data in the one or more images to diagnose the user with cancer, gingivitis, swelling, thrush, dead teeth, gum rot, scurvy, and/or any other illness or medical issue of the mouth. In some embodiments, the diagnostic tool can automatically and dynamically transmit one or more reminders or recommendations to the user. The one or more reminders or recommendations can include reminders or recommendations for the user to brush teeth twice a day, floss, visit a dentist, schedule a dentist appointment and/or any other reminder or recommendation related to mouth hygiene.

In some embodiments, the digital diagnostic tool can transmit the diagnosis of an illness to the system or the telehealth platform. The telehealth platform can use the diagnosis from the digital diagnostic tool to generate an improved diagnosis. The telehealth platform can use user demographic information, a survey response from a user or other users, a body temperature of the user, a resting heart rate of the user, a symptom description of the user, the diagnosis from the digital diagnostic tool, and/or any other information related to the user or the user's health to generate the improved diagnosis.

### Computer Systems

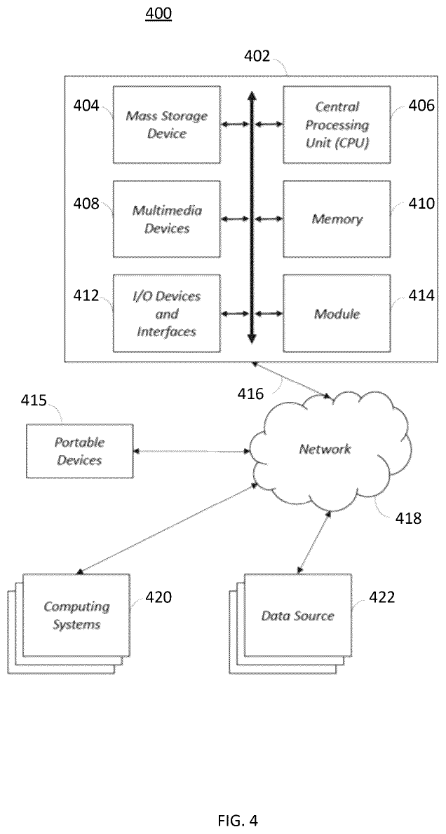

FIG. 4 is a block diagram depicting an embodiment of a computer hardware system configured to run software for implementing the approaches for determining a diagnosis and intervention (e.g., based on a data structure generated for the user) and any systems, methods, and devices disclosed herein. The example computer system 402 is in communication with one or more computing systems 420 and/or one or more data sources 422 via one or more networks 418. While FIG. 4 illustrates an embodiment of a computing system 402, it is recognized that the functionality provided for in the components and modules of computer system 402 may be combined into fewer components and modules, or further separated into additional components and modules.

The computer system 402 can comprise a module 414 that carries out the functions, methods, acts, and/or processes described herein. The module 414 is executed on the computer system 402 by a central processing unit 406 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 402 includes one or more processing units (CPU) 406, which may comprise a microprocessor. The computer system 402 further includes a physical memory 410, such as random-access memory (RAM) for temporary storage of information, a read only memory (ROM) for permanent storage of information, and a mass storage device 404, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 402 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 402 includes one or more input/output (I/O) devices and interfaces 412, such as a keyboard, mouse, touch pad, and printer. The I/O devices and interfaces 412 can include one or more display devices, such as a monitor, which allows the visual presentation of data to a user. More particularly, a display device provides for the presentation of GUIs as application software data, and multi-media presentations, for example. The I/O devices and interfaces 412 can also provide a communications interface to various external devices. The computer system 402 may comprise one or more multi-media devices 408, such as speakers, video cards, graphics accelerators, and microphones, for example.

The computer system 402 may run on a variety of computing devices, such as a server, a Windows server, a Structure Query Language server, a Unix Server, a personal computer, a laptop computer, and so forth. In other embodiments, the computer system 402 may run on a cluster computer system, a mainframe computer system and/or other computing system suitable for controlling and/or communicating with large databases, performing high volume transaction processing, and generating reports from large databases. The computing system 402 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 402 illustrated in FIG. 4 is coupled to a network 418, such as a LAN, WAN, or the Internet via a communication link 416 (wired, wireless, or a combination thereof). Network 418 communicates with various computing devices and/or other electronic devices. Network 418 is communicating with one or more computing systems 420 and one or more data sources 422. The module 414 may access or may be accessed by computing systems 420 and/or data sources 422 through a web-enabled user access point. Connections may be a direct physical connection, a virtual connection, and other connection type. The web-enabled user access point may comprise a browser module that uses text, graphics, audio, video, and other media to present data and to allow interaction with data via the network 418.

Access to the module 414 of the computer system 402 by computing systems 420 and/or by data sources 422 may be through a web-enabled user access point such as the computing systems' 420 or data source's 422 personal computer, cellular phone, smartphone, laptop, tablet computer, e-reader device, audio player, or another device capable of connecting to the network 418. Such a device may have a browser module that is implemented as a module that uses text, graphics, audio, video, and other media to present data and to allow interaction with data via the network 418.

The output module may be implemented as a combination of an all-points addressable display such as a cathode ray tube (CRT), a liquid crystal display (LCD), a plasma display, or other types and/or combinations of displays. The output module may be implemented to communicate with input devices 412 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 402 may comprise a physical or logical connection established between a remote microprocessor and a mainframe host computer for the express purpose of uploading, downloading, or viewing interactive data and databases on-line in real time. The remote microprocessor may be operated by an entity operating the computer system 402, including the client server systems or the main server system, an/or may be operated by one or more of the data sources 422 and/or one or more of the computing systems 420. In some embodiments, terminal emulation software may be used on the microprocessor for participating in the micro-mainframe link.

In some embodiments, computing systems 420 who are internal to an entity operating the computer system 402 may access the module 414 internally as an application or process run by the CPU 406.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 402 may include one or more internal and/or external data sources (for example, data sources 422). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 402 may also access one or more databases 422. The databases 422 may be stored in a database or data repository. The computer system 402 may access the one or more databases 422 through a network 418 or may directly access the database or data repository through I/O devices and interfaces 412. The data repository storing the one or more databases 422 may reside within the computer system 402.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A computer-implemented method, the method comprising:
receiving, from a user device, a first set of indicators associated with a condition experienced by a user;
generating a directed acyclic graph (DAG) for the user, wherein the DAG comprises:
a first layer of nodes that each correspond to an indicator, wherein the first layer of nodes comprises a first node;
a second layer of nodes that each correspond to a cause, wherein the second layer of nodes comprises a second node, wherein each node of the first layer of nodes is connected to each node of the second layer of nodes by an edge, and wherein the edge between the first node of the first layer of nodes and the second node of the second layer of nodes is associated with a probability that the indicator corresponding to the first node is indicative of the cause corresponding to the second node; and
a third layer of nodes that each correspond to a treatment, wherein the third layer of nodes comprises a third node, wherein each node of the second layer of nodes is connected to each node of the third layer of nodes by an edge, and wherein the edge between the second node of the second layer of nodes and the third node of the third layer of nodes is associated with a probability that the treatment corresponding to the third node addresses the cause corresponding to the second node;
traversing the DAG to determine a likely cause;
traversing the DAG to determine at least one treatment for the likely cause; and
generating a custom treatment plan for the user based on:
the at least one treatment for the likely cause; and
a cost function.

2. The computer-implemented method of claim 1, further comprising:
tracking a second set of indicators from the user following the custom treatment plan;
updating the DAG based on the second set of indicators;
traversing the updated DAG to determine an updated cause;
traversing the updated DAG to determine at least one treatment for the updated cause; and
generating an updated treatment plan for the user based on the at least one treatment for the updated cause.

3. The computer-implemented method of claim 1, wherein the directed acyclic graph comprises a recurrent tripartite connected directed acyclic graph.

4. The computer-implemented method of claim 1, wherein traversing the DAG comprises a random sample consensus (RANSAC) approach.

5. The computer-implemented method of claim 1, wherein every node of the DAG is stateful and comprises a presence of an indicator as a percentage.

6. A non-transient computer readable medium containing program instructions for causing a computer to perform a method comprising:
receiving, from a user device, a set of indicators associated with a condition experience by a user;
generating a directed acyclic graph (DAG) for the user, wherein the DAG comprises:
a first layer of nodes that each correspond to an indicator, wherein the first layer of nodes comprises a first node;
a second layer of nodes that each correspond to a cause, wherein the second layer of nodes comprises a second node, wherein each node of the first layer of nodes is connected to each node of the second layer of nodes by an edge, and wherein the edge between the first node of the first layer of nodes and the second node of the second layer of nodes is associated with a probability that the indicator corresponding to the first node is indicative of the cause corresponding to the second node; and
a third layer of nodes that each correspond to a treatment, wherein the third layer of nodes comprises a third node, wherein each node of the second layer of nodes is connected to each node of the third layer of nodes by an edge, and wherein the edge between the second node of the second layer of nodes and the third node of the third layer of nodes is associated with a probability that the treatment corresponding to the third node addresses the cause corresponding to the second node;
traversing the DAG to determine a likely cause;
traversing the DAG to determine at least one treatment for the likely cause; and
generating a custom treatment plan for the user based on:
the at least one treatment for the likely cause; and
a cost function.

7. The non-transient computer readable medium of claim 6, wherein the method further comprises:
tracking a second set of indicators from the user following the custom treatment plan;
updating the DAG based on the second set of indicators;
traversing the updated DAG to determine an updated cause;
traversing the updated DAG to determine at least one treatment for the updated cause; and
generating an updated treatment plan for the user based on the at least one treatment for the updated cause.

8. The non-transient computer readable medium of claim 6, wherein the directed acyclic graph comprises a recurrent tripartite connected directed acyclic graph.

9. The non-transient computer readable medium of claim 6, wherein traversing the DAG comprises a random sample consensus (RANSAC) approach.

10. The non-transient computer readable medium of claim 6, wherein every node of the DAG is stateful and comprises a presence of an indicator as a percentage.
